(19) United States
(12) Patent Application Publication
(10) Pub. No.: US 2025/0285751 A1
(43) Pub. Date: Sep. 11, 2025

(54) Systems, Method, And Computer Program Product For Implementing A Remote Console For Use In Diagnostic Imaging Procedures

(71) Applicant: Bayer Healthcare LLC, Whippany, NJ (US)

(72) Inventors: Sri SHRIRAM, Murrysville, PA (US); David GRIFFITHS, Pittsburgh, PA (US); Sharon STANDISH, Pawleys Island, SC (US); Joanne HOENER, New Kensington, PA (US); Arthur Uber, III, Pittsburgh, PA (US); Linda VAN ROOSMALEN, Gibsonia, PA (US); Kai STRASDAS, Morris Plains, NJ (US); Barry SKIRBLE, Allison Park, PA (US); Gregor Jost, Berlin (DE); Hubertus PIETSCH, Kleinmachnow (DE)

(21) Appl. No.: 18/859,540

(22) PCT Filed: Apr. 27, 2023
(86) PCT No.: PCT/US2023/020190

§ 371 Date: Oct. 24, 2024

Related U.S. Application Data
(60) Provisional application No. 63/336,512, filed on Apr. 29, 2022.

(51) Int. Cl. G16H 40/63; G16H 10/60; G16H 30/40; G16H 40/67

(52) U.S. Cl. CPC G16H 40/63; G16H 10/60; G16H 30/40; G16H 40/67

(57) ABSTRACT

A system, method, and computer program product for implementing a remote console for use in diagnostic imaging may include a user interface and/or at least one processor programmed and/or configured to: receive, from at least one of an injection system including an injector configured to deliver fluid to a patient and an imaging system including a scanner configured to scan the patient, procedure data associated with a procedure for the patient, at least one of the injection system and the imaging system including at least one further user interface different than the user interface, and the injection system being in communication with the imaging system; provide, via the user interface, a display, the display being generated based on the procedure data; receive, via the user interface, user input; and control, based on the user input, an operation of at least one of the injection system and the imaging system.

### CROSS-REFERENCE TO RELATED APPLICATIONS

This application claims the benefit of U.S. Provisional Application No. 63/336,512, filed Apr. 29, 2022, the disclosure of which is hereby incorporated by reference in its entirety.

### BACKGROUND

### 1. Field

This disclosure relates generally to diagnostic imaging, and in some non-limiting embodiments or aspects, to a remote console for use in diagnostic imaging procedures.

### 2. Technical Considerations

Injecting contrast media into bloodstreams of patients enables visualization of various pathologies through X-ray, computed tomography, magnetic resonance, or other medical-imaging modalities. Contrast delivery is more effective and efficient using a medical device called a “power fluid injector system” that can be programmed to deliver specific amounts of contrast agent at specific flow rates with specific timing in relation to the image acquisition.

### SUMMARY

Accordingly, provided are improved systems, devices, products, apparatus, and/or methods for implementing a remote console for use in diagnostic imaging procedures.

According to some non-limiting embodiments or aspects, provided is a system, including: a user interface; and at least one processor programmed and/or configured to: receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; provide, via the user interface, a display, wherein the display is generated based on the procedure data; receive, via the user interface, user input; and control, based on the user input, an operation of at least one of the injection system and the imaging system.

In some non-limiting embodiments or aspects, the procedure data includes at least one of the following: at least one image captured by a camera associated with at least one of the injection system and the imaging system; information received via the at least one further user interface; sensor data captured by at least one sensor associated with at least one of the injection system and the imaging system; or any combination thereof.

In some non-limiting embodiments or aspects, the procedure data includes the at least one image, and wherein the at least one image includes at least one of the following: one or more images of the patient; one or more images of an operator of at least one of the injection system and the imaging system; one or more images of an IV access location on the patient; one or more images of the injector; one or more images of the scanner; or any combination thereof.

In some non-limiting embodiments or aspects, the procedure data includes the information received via the at least one further user interface, and wherein the information received via the at least one further user interface includes at least one of the following: an imaging prescription; a pre-procedure checklist; an indication of patient consent; an adverse reaction checklist; at least one of a selection, a programming, and a modification of an injection protocol; at least one of a selection, a programming, and a modification of an imaging protocol; a request to initiate the operation; a request to end the procedure; information captured at an end of the procedure; information for a secondary capture; information for a billing procedure; or any combination thereof.

In some non-limiting embodiments or aspects, the procedure data includes the sensor data captured by the at least one sensor associated with the at least one of the injection system and the imaging system, and wherein the at least one sensor includes at least one of the following sensors: a flow sensor; a temperature sensor; an accelerometer; a vibration sensor; a strain gauge; a motor current sensor; an optical sensor; the scanner; or any combination thereof, and wherein the procedure data includes at least one of the following parameters determined based on the sensor data: a movement associated with the patient; a sound associated with the patient; a communication state between the scanner and the injector; one or more images of the patient captured by the scanner; an amount of noise associated with the one or more images of the patient captured by scanner; an enhancement or saturation level associated with the one or more images of the patient captured by scanner; a flow rate during one or more fluid injections by the injector; a duration of the one or more fluid injections by the injector; a volume pumped and/or delivered during the one or more injections by the injector; an achieved pressure of the one or more injections by the injector; a flow rate; a imaging signal from a region of interest at a time or over a length of time; an ECG gating sequence; a pulse of a patient; or any combination thereof.

In some non-limiting embodiments or aspects, the sensor data is captured by the at least one sensor during at least one of the following: a setup of at least one of the injector and the scanner; an arming of the injector; a period of time when the injector is armed; a keep vein open (KVO) operation by the injector; a delivery of a test bolus and/or a tracking bolus to the patient by the injector; a delivery of a contrast media to the patient by the injector; a period of time after delivery of the contrast media to the patient; a delivery of a saline flush to the patient by the injector; a period of time after delivery of the saline flush to the patient; a scanning of the patient by the scanner; or any combination thereof.

In some non-limiting embodiments or aspects, the display includes an alert, and wherein at least one of the injection system and the imaging system generates the alert based on at least one of the following: the sensor data; the user input; patient data associated with the patient; or any combination thereof.

In some non-limiting embodiments or aspects, the procedure data includes patient data including at least one of the following parameters associated with the patient: an age; a weight; a height; a body mass index; a cardiac output; a risk or probability of an adverse event; an adverse event; an estimated or measured glomerular filtration rate (eGFR); a prior injection history including one or more reactions to contrast media; a location of a patient IV access point; a quality of the patient IV access point; or any combination thereof.

In some non-limiting embodiments or aspects, the display includes an alert, and wherein at least one of the injection system and the imaging system generates the alert based on the patient data before arming the injector for the procedure.

In some non-limiting embodiments or aspects, the at least one processor is further programmed and/or configured to retrieve information associated with at least one of the procedure and the patient from at least one of the following: an electronic medical record (EMR) system; a radiology information system (RIS); a picture archiving and communication system (PACS); a hospital information system (HIS); a hospital enterprise information system; a cross location network; or any combination thereof.

In some non-limiting embodiments or aspects, the user input includes at least one of a selection, a programming, and a modification of at least one of an injection protocol and an imaging protocol.

In some non-limiting embodiments or aspects, the display includes a prompt to confirm the operation of the at least one of the injection system and the imaging system, and wherein the at least one processor is programmed and/or configured to control the operation of the at least one of the injection system and the imaging system by: controlling the at least one of the injection system and the imaging system such that the at least one of the injection system and the imaging system is prevented from performing the operation until a response to the prompt is received via the user interface.

In some non-limiting embodiments or aspects, the operation includes at least one of the following operations: changing a display of the at least one further user interface; receiving further user input via the at least one further user interface; setting an injection protocol at the injector; setting an imaging protocol at the scanner; initiating a loading process with the injector; initiating a data capture with the injector; arming the injector; initiating a keep vein open (KVO) procedure with the injector; initiating a saline test injection with the injector; initiating a drug injection with the injector; initiating a contrast media test injection with the injector; initiating a contrast media injection with the injector; initiating a saline flush injection with the injector; initiating a scan with the scanner; aborting the test injection with the injector; aborting the contrast media injection with the injector; aborting the saline flush injection with the injector; aborting the scan with the scanner; ending the procedure for the patient; or any combination thereof.

In some non-limiting embodiments or aspects, the operation includes at least one of delivering fluid to the patient with the injector and scanning the patient with the scanner, and wherein the at least one processor is programmed and/or configured to control the operation of the at least one of the injection system and the imaging system by: controlling the at least one of the injection system and the imaging system such that the at least one of the injection system and the imaging system at least one of modifies and aborts the operation.

In some non-limiting embodiments or aspects, the procedure data includes one or more images of the patient captured by the scanner, and wherein the at least one processor is programmed and/or configured to process the one or more images to select a subset of the one or more images, and wherein the display is generated based on the selected subset of the one or more images.

In some non-limiting embodiments or aspects, the scanner includes at least one of the following: a magnetic resonance (MR) scanner; a computed tomography (CT) scanner; a positron emission tomography/computed tomography (PET/CT) scanner; a positron emission tomography/magnetic resonance (PET/MR) scanner; a positron emission tomography (PET) scanner; a single-photon emission computerized tomography (SPECT) scanner; an ultrasound system, an infrared imaging system, a photo-acoustic imaging system, or any combination thereof.

In some non-limiting embodiments or aspects, the injection system, the imaging system, and the at least one further user interface are located at a procedure location, and wherein the user interface is located at a support location different than the procedure location.

In some non-limiting embodiments or aspects, the procedure location includes at least one of the following locations: a hospital, an imaging center, a mobile imaging trailer, an outpatient medical facility, a nursing care center, a vehicle, or any combination thereof.

In some non-limiting embodiments or aspects, the user input includes at least one of the following: a response to a prompt to confirm the operation of the at least one of the injection system and the imaging system; an instruction to the at least one of the injection system and the imaging system to initiate, modify, and/or perform the operation of the at least one of the injection system and the imaging system; or any combination thereof.

In some non-limiting embodiments or aspects, the display includes at least one alert, and wherein the at least one alert includes at least one of the following: an indication that the procedure is complete; an indication of whether the procedure executed correctly; an indication that an operator appears hesitant during programming; a real-time display of measured parameters associated with a fluid injection; an indication of a detected adverse event; an indication of patient discomfort, an indication of patient motion, an indication of a quality of one or more images of the patient captured by the scanner; or any combination thereof.

In some non-limiting embodiments or aspects, the at least one processor is programmed and/or configured to: receive, from the at least one of the injection system and the imaging system via the at least one further user interface, authentication data associated with a user account; restrict, based on an authorization level associated with the user account, one or more operations of at least one of the injection system and the imaging system from being initiated and/or modified via the at least one further user interface by the user account, wherein the authorization level associated with the user account is determined based on the authentication data.

In some non-limiting embodiments or aspects, the authorization level is further determined based on at least one of the following: a number of procedures associated with the user account; a quality, experience, and/or other rating associated with the user account; a type and/or an aspect of the procedure; a type of the patient; or any combination thereof.

In some non-limiting embodiments or aspects, the authorization level associated with the user account is selected from one of the following authorization levels: a first authorization level at which the user account is restricted from modifying at least one of an injection protocol and a scanning protocol; a second authorization level at which the user account is restricted from modifying a first subset of parameters of the at least one of the injection protocol and the scanning protocol and enabled to modify a second subset of parameters of the at least one of the injection protocol and the scanning protocol; a third authorization level at which the user account is enabled to modify each parameter of the at least one of the injection protocol and the scanning protocol.

According to some non-limiting embodiments or aspects, provided is a method including: providing, with at least one processor, a user interface; receiving, with the at least one processor, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; providing, with the at least one processor, via the user interface, a display, wherein the display is generated based on the procedure data; receiving, with the at least one processor, via the user interface, user input; and controlling, with the at least one processor, based on the user input, an operation of at least one of the injection system and the imaging system.

According to some non-limiting embodiments or aspects, provided is a computer program product comprising at least one non-transitory computer-readable medium including program instructions that, when executed by at least one processor, cause the at least one processor to: provide a user interface; receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; provide, via the user interface, a display, wherein the display is generated based on the procedure data; receive, via the user interface, user input; and control, based on the user input, an operation of at least one of the injection system and the imaging system.

Further non-limiting embodiments or aspects are set forth in the following numbered clauses:

Clause 1. A system, comprising: a user interface; and at least one processor programmed and/or configured to: receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; provide, via the user interface, a display, wherein the display is generated based on the procedure data; receive, via the user interface, user input; and control, based on the user input, an operation of at least one of the injection system and the imaging system.

Clause 2. The system of clause 1, wherein the procedure data includes at least one of the following: at least one image captured by a camera associated with at least one of the injection system and the imaging system; information received via the at least one further user interface; sensor data captured by at least one sensor associated with at least one of the injection system and the imaging system; or any combination thereof.

Clause 3. The system of any of clauses 1 or 2, wherein the procedure data includes the at least one image, and wherein the at least one image includes at least one of the following: one or more images of the patient; one or more images of an operator of at least one of the injection system and the imaging system; one or more images of an IV access location on the patient; one or more images of the injector; one or more images of the scanner; or any combination thereof.

Clause 4. The system of any of clauses 1-3, wherein the procedure data includes the information received via the at least one further user interface, and wherein the information received via the at least one further user interface includes at least one of the following: an imaging prescription; a pre-procedure checklist; an indication of patient consent; an adverse reaction checklist; at least one of a selection, a programming, and a modification of an injection protocol; at least one of a selection, a programming, and a modification of an imaging protocol; a request to initiate the operation; a request to end the procedure; information captured at an end of the procedure; information for a secondary capture; information for a billing procedure; or any combination thereof.

Clause 5. The system of any of clauses 1-4, wherein the procedure data includes the sensor data captured by the at least one sensor associated with the at least one of the injection system and the imaging system, and wherein the at least one sensor includes at least one of the following sensors: a flow sensor; temperature sensor; an accelerometer; a vibration sensor; a strain gauge; a motor current sensor; an optical sensor; the scanner; or any combination thereof, and wherein the procedure data includes at least one of the following parameters determined based on the sensor data: a movement associated with the patient; a sound associated with the patient; a communication state between the scanner and the injector; one or more images of the patient captured by the scanner; an amount of noise associated with the one or more image of the patient captured by scanner; an enhancement or saturation level associated with the one or more images of the patient captured by scanner; a flow rate during one or more fluid injections by the injector; a duration of the one or more fluid injections by the injector; a volume pumped and/or delivered during the one or more injections by the injector; an achieved pressure of the one or more injections by the injector; a flow rate; an imaging signal from a region of interest at a time or over a length of time; an ECG gating sequence; a pulse of a patient; or any combination thereof.

Clause 6. The system of any of clauses 1-5, wherein the sensor data is captured by the at least one sensor during at least one of the following: a setup of at least one of the injector and the scanner; an arming of the injector; a period of time when the injector is armed; a keep vein open (KVO) operation by the injector; a delivery of a test bolus and/or a tracking bolus to the patient by the injector; a delivery of a contrast media to the patient by the injector; a period of time after delivery of the contrast media to the patient; a delivery of a saline flush to the patient by the injector; a period of time after delivery of the saline flush to the patient; a scanning of the patient by the scanner; or any combination thereof.

Clause 7. The system of any of clauses 1-6, wherein the display includes an alert, and wherein at least one of the injection system and the imaging system generates the alert based on at least one of the following: the sensor data; the user input; patient data associated with the patient; or any combination thereof.

Clause 8. The system of any of clauses 1-7, wherein the procedure data includes patient data including at least one of the following parameters associated with the patient: an age; a weight; a height; a body mass index; a cardiac output; a risk or probability of an adverse event; an adverse event; an estimated or measured glomerular filtration rate (eGFR); a prior injection history including one or more allergic reactions to contrast media; a location of a patient IV access point; a quality of the patient IV access point; or any combination thereof.

Clause 9. The system of any of clauses 1-7, wherein the display includes an alert, and wherein at least one of the injection system and the imaging system generates the alert based on the patient data before arming the injector for the procedure.

Clause 10. The system of any of clauses 1-8, wherein the at least one processor is further programmed and/or configured to retrieve information associated with at least one of the procedure and the patient from at least one of the following: an electronic medical record (EMR) system; a radiology information system (RIS); a picture archiving and communication system (PACS); a hospital information system (HIS); a hospital enterprise information system; a cross location network; or any combination thereof.

Clause 11. The system of any of clauses 1-9, wherein the user input includes at least one of a selection, a programming, and a modification of at least one of an injection protocol and an imaging protocol.

Clause 12. The system of any of clauses 1-10, wherein the display includes a prompt to confirm the operation of the at least one of the injection system and the imaging system, and wherein the at least one processor is programmed and/or configured to control the operation of the at least one of the injection system and the imaging system by: controlling the at least one of the injection system and the imaging system such that the at least one of the injection system and the imaging system is prevented from performing the operation until a response to the prompt is received via the user interface.

Clause 13. The system of any of clauses 1-11, wherein the operation includes at least one of the following operations: changing a display of the at least one further user interface; receiving further user input via the at least one further user interface, setting an injection protocol at the injector; setting an imaging protocol at the scanner; initiating a loading process with the injector; initiating a data capture with the injector; arming the injector; initiating a keep vein open (KVO) procedure with the injector; initiating a saline test injection with the injector; initiating a drug injection with the injector; initiating a contrast media test injection with the injector; initiating a contrast media injection with the injector; initiating a scan with the scanner; aborting the test injection with the injector; aborting the contrast media injection with the injector; initiating a saline flush injection with the injector; aborting the saline flush injection with the injector; aborting the scan with the scanner; initiating a saline flush injection with the injector; aborting the saline flush injection with the injector; ending the procedure for the patient; or any combination thereof.

Clause 14. The system of any of clauses 1-13, wherein the operation includes at least one of delivering fluid to the patient with the injector and scanning the patient with the scanner, and wherein the at least one processor is programmed and/or configured to control the operation of the at least one of the injection system and the imaging system by: controlling the at least one of the injection system and the imaging system such that the at least one of the injection system and the imaging system at least one of modifies and aborts the operation.

Clause 15. The system of any of clauses 1-14, wherein the procedure data includes one or more images of the patient captured by the scanner, and wherein the at least one processor is programmed and/or configured to process the one or more images to select a subset of the one or more images, and wherein the display is generated based on the selected subset of the one or more images.

Clause 16. The system of any of clauses 1-15, wherein the scanner includes at least one of the following: a magnetic resonance (MR) scanner; a computed tomography (CT) scanner; a positron emission tomography/computed tomography (PET/CT) scanner; a positron emission tomography/magnetic resonance (PET/MR) scanner; a single-photon emission computerized tomography (SPECT) scanner; an ultrasound system, an infrared imaging system, a photo-acoustic imaging system, or any combination thereof.

Clause 17. The system of any of clauses 1-16, wherein the injection system, the imaging system, and the at least one further user interface are located at a procedure location, and wherein the user interface is located at a support location different than the procedure location.

Clause 18. The system of any of clauses 1-17, wherein the procedure location includes at least one of the following locations: a hospital, an imaging center, a mobile imaging trailer, a vehicle, or any combination thereof.

Clause 19. The system of any of clauses 1-18, wherein the user input includes at least one of the following: a response to a prompt to confirm the operation of the at least one of the injection system and the imaging system; an instruction to the at least one of the injection system and the imaging system to initiate, modify, and/or perform the operation of the at least one of the injection system and the imaging system; or any combination thereof.

Clause 20. The system of any of clauses 1-19, wherein the display includes at least one alert, and wherein the at least one alert includes at least one of the following: an indication that the procedure is complete; an indication of whether the procedure executed correctly; an indication that an operator appears hesitant during programming; a real-time display of measured parameters associated with a fluid injection; an indication of a detected adverse event; an indication of a quality of one or more images of the patient captured by the scanner; or any combination thereof.

Clause 21. The system of any of clauses 1-20, wherein the at least one processor is programmed and/or configured to: receive, from the at least one of the injection system and the imaging system via the at least one further user interface, authentication data associated with a user account; restrict, based on an authorization level associated with the user account, one or more operations of at least one of the injection system and the imaging system from being initiated and/or modified via the at least one further user interface by the user account, wherein the authorization level associated with the user account is determined based on the authentication data.

Clause 22. The system of any of clauses 1-21, wherein the authorization level is further determined based on at least one of the following: a number of procedures associated with the user account; a quality, experience, and/or other rating associated with the user account; a type and/or an aspect of the procedure; a type of the patient; or any combination thereof.

Clause 23. The system of any of clauses 1-22, wherein the authorization level associated with the user account is selected from one of the following authorization levels: a first authorization level at which the user account is restricted from modifying at least one of an injection protocol and a scanning protocol; a second authorization level at which the user account is restricted from modifying a first subset of parameters of the at least one of the injection protocol and the scanning protocol and enabled to modify a second subset of parameters of the at least one of the injection protocol and the scanning protocol; a third authorization level at which the user account is enabled to modify each parameter of the at least one of the injection protocol and the scanning protocol.

Clause 24. A method including: providing, with at least one processor, a user interface; receiving, with the at least one processor, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; providing, with the at least one processor, via the user interface, a display, wherein the display is generated based on the procedure data; receiving, with the at least one processor, via the user interface, user input; and controlling, with the at least one processor, based on the user input, an operation of at least one of the injection system and the imaging system.

Clause 25. A computer program product comprising at least one non-transitory computer-readable medium including program instructions that, when executed by at least one processor, cause the at least one processor to: provide a user interface; receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system; provide, via the user interface, a display, wherein the display is generated based on the procedure data; receive, via the user interface, user input; and control, based on the user input, an operation of at least one of the injection system and the imaging system.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS AND APPENDICES

Additional advantages and details are explained in greater detail below with reference to the exemplary embodiments that are illustrated in the accompanying schematic figures, in which:

FIG. 1A is a diagram of non-limiting embodiments or aspects of an environment in which systems, devices, products, apparatus, and/or methods, described herein, may be implemented;

FIG. 1B is a diagram of non-limiting embodiments or aspects of components of one or more devices and/or one or more systems of FIG. 1A;

FIG. 2 is a diagram of non-limiting embodiments or aspects of components of one or more devices and/or one or more systems of FIGS. 1A and 1B;

FIG. 3 is a flowchart of non-limiting embodiments or aspects of a process for implementing a remote console for use in diagnostic imaging procedures;

FIGS. 4A-4C illustrate an implementation of non-limiting embodiments or aspects of a user interface of a remote console for use in diagnostic imaging procedures; and

FIG. 5 is a diagram of an implementation of non-limiting embodiments or aspects of a process for implementing a remote console for use in diagnostic imaging procedures.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the term “communication” may refer to the reception, receipt, transmission, transfer, provision, and/or the like, of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc. In some non-limiting embodiments or aspects, there may be multiple thresholds associated with a single parameter or measurement, for example, during a fluid delivery, a pressure higher than a first threshold may indicate a blocked fluid path, whereas a pressure lower than a second threshold may indicate a leak and/or broken fluid path.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a PDA, and/or other like devices. A computing device may also be a desktop computer or other form of non-mobile computer.

As used herein, the term “mobile device” may refer to one or more portable electronic devices configured to communicate with one or more networks. As an example, a mobile device may include a cellular phone (e.g., a smartphone or standard cellular phone), a portable computer (e.g., a tablet computer, a laptop computer, etc.), a wearable device (e.g., a watch, pair of glasses, lens, clothing, and/or the like), a personal digital assistant (PDA), and/or other like devices. The terms “client device” and “user device,” as used herein, refer to any electronic device that is configured to communicate with one or more servers or remote devices and/or systems. A client device or user device may include a mobile device, a network-enabled appliance (e.g., a network-enabled television, refrigerator, thermostat, and/or the like), a computer, an injection system, and/or any other device or system capable of communicating with a network.

As used herein, the term “server” and/or “processor” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, injectors, mobile devices, etc.) directly or indirectly communicating in the network environment may constitute a “system.” Reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “user interface” or “graphical user interface” refers to a generated display, such as one or more graphical user interfaces (GUIs) with which a user may interact, either directly or indirectly (e.g., through a keyboard, mouse, touchscreen, etc.).

As used herein, the terms “scan” and “scanning”, for example, by a scanner, refer to acquiring and/or generating one or more pieces of information used to create one or more images and/or measurements of the patient, such as a CT scan or image, and/or the like.

Non-limiting embodiments or aspects of the present disclosure may provide one or more of the following benefits to users thereof, their coworkers, their employers, their patients, and/or their patients' caregivers. They may enable less experienced users (e.g., technologists, radiographers, nurses, medical assistants, etc.) to complete a procedure for which these less experienced users have little or no experience. A single more proficient user may help or oversee multiple less experienced users. Every technologist may be enabled to work like an expert. They may enable local and/or “on the job” training of users. They may serve as a safety net for new users, reducing their stress and the reducing the need for local supervision. They may provide ongoing training in the course of doing their normal work. They may promote standardization and protocol optimization. They may help ensure that imaging procedures have sufficient diagnostic quality. They may help maximize throughput. They may help minimize repeat scans if not appropriate. They may help monitor for patient safety by having more checks that a single user could provide. They may provide input to help a user decide if it is ok to attempt a repeat scan. They may promote reuse of the same procedure next time if it has been successful, promoting learning. They may enable the virtual imaging specialists to steer or supervise multiple scanners at geographically different sites which would otherwise require travel and take time. They may monitor performance, for example throughput, human factors related to all the devices and systems, and overall procedure quality. They may optimize integration of scanner and injector consoles (e.g., merging or combining of the two separate user interfaces, etc.). They may reduce duplicative data entry. They may help address a shortage of healthcare workers. They may help redistribute the tasks that were previously performed at the scanner. They may enable focus on core tasks by each person involved. They may increase patient convenience and imaging quality. They may facilitate the sharing of in-house expertise. They may facilitate customer training from two different parties, for example, the injector supplier and the imager supplier, at the same time. They may enable better leveling or distributing of a workload, for example, the most experienced person no longer has to receive many of the most difficult cases. They may reduce costs by not sending a dedicated/expert user from one geographic site to another. They may increase patient satisfaction and save patients time as patients can be scanned nearer where they are and not have to travel to where the expertise is located. They may better utilize multiple imagers, for example, because protocols will not be limited by the expertise of the local user. They may help keep experts from being spread too thin and being overworked. They may increase experts' job satisfaction as they are able to do higher-level tasks and not all the lower-level tasks. They may facilitate real-time consultation on outlier or high-risk patients and on outlier or new procedures. They may enable local technologist and Virtual Imaging Specialist to have the same situational view of the procedure. They may enable options for either local or remote control of the start of the procedures. They may provide alerts and/or warnings if a procedure has off-label settings (e.g., full dose vs half dose in MR, etc.). They may provide step-by-step protocols for lower-skilled technologists. They may provide a “4 eyes check” in relation to adverse event handling. They may help facilitate and centralize adverse event reporting. They may combine scanner and injector interfaces to provide a more unified user interface experience. They may enable the local technologist to focus more on the patient and the patient's comfort and safety. They may enable the local technologist to focus on the patient and reduce patient walk-outs mid-procedure. They may provide for contextual appearance or inclusion of specific injection protocols for specific contrast enhanced scan protocols. They may enable service personnel training or cross training from two parties (e.g., injector and imager organizations) at same time. They may enable remote screening of patients. They may enable local nurses who focus on the patient, and remote techs who focuses on the scanner and the protocol. They may enable training without the need for travel which provides a sustainability benefit. They may be needed more and more as the number and expertise needed to operate imaging systems are growing. They may enable access to remote expertise to answer patients' tougher questions. They may provide guidance across multiple injector systems, for example, injectors from Bayer, Ulrich, Nemoto, and others. They may enable remote consultation with patients to improve compliance and cooperation, thereby reducing “scanxiety”. These herein mentioned benefits and features of non-limiting embodiments or aspects of the present disclosure may be achieved, for example, by providing for a less experienced user to request help from, an interaction with, and/or a consultation with a more experienced user. This can allow the more experienced user to observe and/or suggest/intervene in the use of the system by the less experienced user, and/or for at least one of an imaging system, an injection system, a procedure system, a support system, a local system, and/or additional devices thereof to inform or alert either user that an interaction and/or consultation may be beneficial.

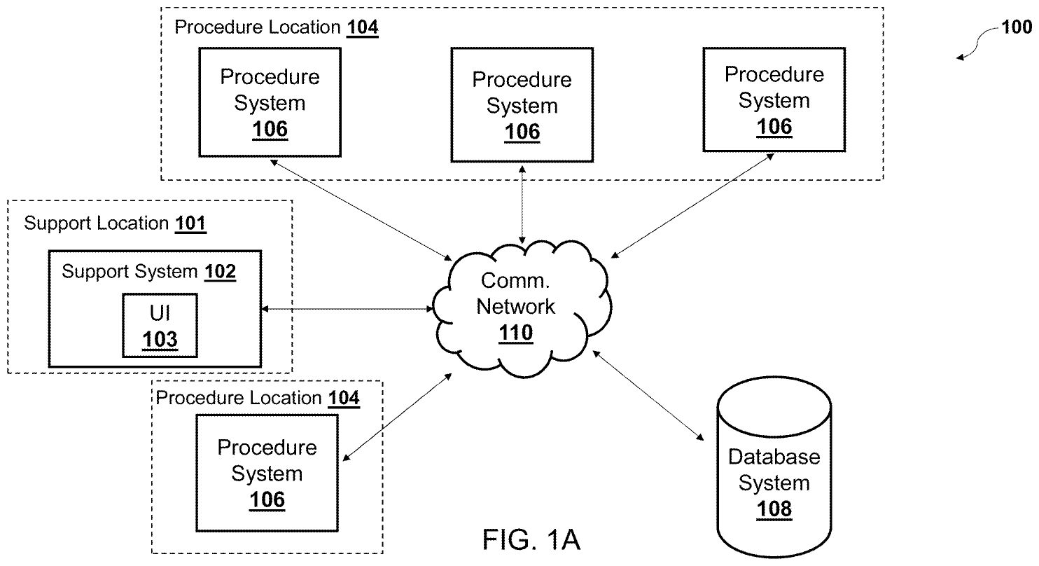

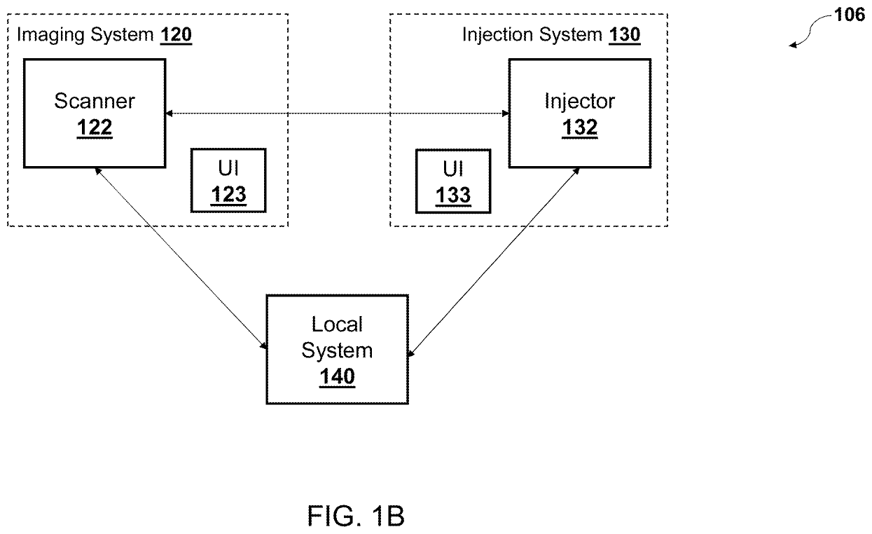

Referring now to FIG. 1A, FIG. 1A is a diagram of an example environment 100 in which systems, devices, methods, and/or products described herein, may be implemented. As shown in FIG. 1A, environment 100 may include a support location 101 including support system 102, one or more procedure locations 104 including one or more procedure systems 106, database system 108, and/or communication network 110.

Support system 102 may include one or more devices capable of receiving data and/or information from and/or communicating data and/or information to one or more procedure systems 106 at one or more procedure locations 104 and/or database system 108 (e.g., via communication network 110, etc.). For example, support system 102 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices.

Support system 102 may include user interface 103 (e.g., an input component 210, an output component 212, etc.). Support location 101 including support system 102 that includes user interface 103 may include a remote console control room, an isolation room, a home office, and/or the like.

A procedure location 104 may include one or more procedure systems 106 (e.g., a single procedure system 106, a plurality of procedure systems 106, etc.). For example, a procedure location 104 may include a hospital, an imaging center, a mobile imaging trailer, an emergency vehicle (e.g., an ambulance, etc.), a mobile imaging device (e.g., Hyperfine's Swoop® Portable MR Imaging System™, etc.), a mobile CT device, and/or the like. As an example, environment 100 may include a plurality of procedure locations 104 including a plurality of procedure systems 106. In such an example, the plurality of procedure locations 104 may include a plurality of different locations (e.g., a hospital, an imaging center, an ambulance, etc.), and/or support location 101 may include a different location than the plurality of procedure locations 104.

A procedure system 106 may include one or more devices capable of receiving data and/or information from and/or communicating data and/or information to support system 102 and/or database system 108 (e.g., via communication network 110, etc.). For example, a procedure system 106 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices.

Referring now also to FIG. 1B, FIG. 1B is a diagram of example components of a procedure system 106. As shown in FIG. 6, a procedure system may include imaging system 120 including scanner 122, injection system 130 including injector 132, and/or local system 140. Imaging system 120 including scanner 122, injection system 130 including injector 132, and/or local system 140 may interconnect (e.g., establish a connection to communicate, etc.) via wired connections, wireless connections, or a combination of wired and wireless connections. A procedure system 106 may also include devices and/or systems not expressly part of the scanner 122 or injector 132 that are also part of selected procedures, for example patient monitoring equipment, anesthesia equipment, and/or similar healthcare equipment. Such equipment may be in communication with any systems associated with the procedure system 106, the local system 140, and/or any support system 102. In some non-limiting embodiments or aspects, one or more additional or auxiliary devices (e.g., one or more sensors, patient monitors, infusion pumps, anesthesia equipment, biopsy guidance systems, etc.), each of which may include a user interface itself, may be connected to and/or in communication with scanner 122, injector 132, and/or local system 140.

Imaging system 120 may include one or more devices, software, and/or hardware configured to set up imaging protocols (e.g., a scan time, etc.) and acquire non-contrast and contrast-enhanced scans of a patient. For example, imaging system 120 may include scanner 122 configured to image a patient according to one or more imaging protocols. As an example, imaging system 120 may include a magnetic resonance imaging (MRI) system; a computed tomography (CT) system; a positron emission tomography/computed tomography (PET/CT) system; a positron emission tomography/magnetic resonance (PET/MR) system; a positron emission tomography (PET) system; a single-photon emission computed tomography (SPECT) system; an ultrasound system; a radiopharmaceutical therapy (RPT) system, an infrared imaging system, a photo-acoustic imaging system, and/or the like. In such an example, scanner 122 may include a magnetic resonance (MR) scanner; a computed tomography (CT) scanner; a positron emission tomography/computed tomography (PET/CT) scanner; a positron emission tomography/magnetic resonance (PET/MR) scanner; a positron emission tomography (PET) scanner; a single-photon emission computerized tomography (SPECT) scanner; an ultrasound system; a radiopharmaceutical therapy (RPT) scanner; an infrared imaging scanner, a photo-acoustic imaging scanner, and/or the like. As an example, imaging system 120 may include an imaging system as described in U.S. patent application Ser. No. 16/710,118, filed on Dec. 11, 2019, the entire contents of which is hereby incorporated by reference. In some non-limiting embodiments or aspects, imaging system 120 includes Siemens Syngo remote console, Siemens Healthineers' Somatom Go CT System, General Electric's Signa MR System, and/or the like.

In some non-limiting embodiments or aspects, imaging system 120 includes a system for standardized MRI examinations with patient-centric scan workflow adaptations as described by U.S. patent application Ser. No. 17/458,753, filed on Aug. 27, 2021, the disclosure of which is incorporated herein by reference in its entirety.

Injection system 130 may include one or more devices, software, and/or hardware configured to set up one or more injection protocols and deliver one or more fluids (e.g., saline, a contrast agent, a stress agent, etc.) to a patient according to one or more injection protocols. An injection protocol commonly includes one or more phases, with each phase specifying the fluid or fluids and optionally fluid concentration to be injected, and two of the flow rate, volume, and duration of that phase of the injection (e.g., because volume injected=flow rate×duration, and/or the integral of flow rate over injection duration for more complicated protocols, there are only two independent variables out of those three parameters). Other injection parameters which may be different for different phases or constant for all phases may include at least one of a ratio of fluids injected, for example, percent of contrast in a total fluid flow, pressure limits, pressure limit behaviors, flow rate limits, occlusion indications, or any combination thereof. Some injectors may be configured to have a time varying value of one, some, or all of the injection parameters. For example, injection system 130 may include injector 132 configured to deliver one or more fluids (e.g., saline, a contrast agent, etc.) to a patient according to one or more injection protocols. As an example, injection system 130 may include a contrast injection system as described in U.S. Pat. No. 6,643,537 and/or 7,937,134 and/or as described in published International Application No. WO2019046299A1, the entire contents of each of which is hereby incorporated by reference. As an example, injection system 130 may include the MEDRAD® Stellant FLEX CT Injection System, the MEDRAD® MRXperion MR Injection System, the MEDRAD® Mark 7 Arterion Injection System, the MEDRAD® Intego PET Infusion System, the MEDRAD® Spectris Solaris EP MR Injection System, the MEDRAD® Stellant CT Injection System With Certegra® Workstation, and/or the like.

In some non-limiting embodiments or aspects, imaging system 120, injection system 130, and/or local system 140 include and/or receive sensor data from one or more additional devices, such as one or more sensors, patient monitors, infusion pumps, anesthesia equipment, biopsy guidance systems, and/or the like, which may, for example, include sensors capable of receiving, determining, measuring, and/or sensing sensor data associated with the patient, scanner 122, injector 132, and/or the operator. For example, imaging system 120, injection system 130, and/or local system 140 may include and/or receive sensor data from at least one of the following sensors: an image capture device; an accelerometer; a strain gauge; a global positioning system (GPS); a skin resistivity or conductance sensor; a heart rate monitor; a microphone; a thermal or temperature sensor; a pulse oximeter; a hydration sensor; a dosimeter; an ultrasound sensor; an acoustic sensor; one or more electrodes configured to measure at least one of a tissue impedance, an electromyogram (EMG), and an electrocardiogram (ECG); a microwave sensor; a mechanical impedance sensor; a chemical sensor; a force or pressure sensor; a flow sensor; an accelerometer; a vibration sensor; a motor current sensor; an optical sensor; a scale for patient weight; scanner 122; injector 132; or any combination thereof. In some non-limiting embodiments or aspects, imaging system 120, injection system 130, and/or local system 140 may include and/or receive sensor data from one or more sensors described by International Patent Application Publication No, WO2021222771A1, published Nov. 4, 2021, which was filed as International Patent Application No. PCT/US2021/030210 on Apr. 30, 2021, the entire contents of which is hereby incorporated by reference.

Local system 140 may include one or more devices capable of receiving data and/or information from and/or communicating data and/or information to imaging system 120 and/or injection system 130. For example, local system 140 may include a computing device, such as a one or more computers, portable computers (e.g., tablet computers, etc.), mobile devices (e.g., cellular phones, smartphones, wearable devices, such as watches, glasses, lenses, and/or clothing, PDAs, and/or the like), a server, a group of servers, and/or other like devices. As an example, local system 140 may be implemented by and/or associated with a hospital, an imaging center, a mobile imaging trailer, an emergency vehicle (e.g., an ambulance, etc.), a mobile imaging device (e.g., Hyperfine's Swoop® Portable MR Imaging System™, etc.), a portable or mobile CT device, and/or the like. In some non-limiting embodiments or aspects, local system 140 may include one or more additional or auxiliary devices (e.g., one or more sensors, patient monitors, infusion pumps, anesthesia equipment, biopsy guidance systems, etc.), each of which may include a user interface itself, and which may be connected to and/or in communication with scanner 122 and/or injector 132.

Database system 108 may include one or more devices capable of receiving information and/or data from one or more procedure systems 106 at one or more procedure locations 104 and/or support system 102 (e.g., via communication network 110, etc.) and/or communicating information and/or data to one or more procedure systems 106 at one or more procedure locations 104 and/or support system 102 (e.g., via communication network 110, etc.). For example, database system 108 may include one or more computing systems including one or more processors (e.g., one or more computing devices, one or more server computers, one or more mobile computing devices, etc.). As an example, database system 108 may include an electronic medical record (EMR) system; a radiology information system (RIS); a picture archiving and communication system (PACS); a hospital information system (HIS); a hospital enterprise information system; an insurance or payment information system, a national health information system, a cross location network; or any combination thereof.

Communication network 110 may include one or more wired and/or wireless networks. For example, communication network 110 may include a cellular network (e.g., a long-term evolution (LTE) network, a third generation (3G) network, a fourth generation (4G) network, a fifth generation (5G) network, a sixth generation (6G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, a short range wireless communication network (e.g., a Bluetooth network, a near field communication (NFC) network, etc.) and/or the like, and/or a combination of these or other types of networks.

The number and arrangement of devices and systems shown in FIGS. 1A and 1B is provided as an example. There may be additional devices and/or systems, fewer devices and/or systems, different devices and/or systems, or differently arranged devices and/or systems than those shown in FIGS. 1A and 1B. Furthermore, two or more devices and/or systems shown in FIGS. 1A and 1B may be implemented within a single device and/or system, or a single device and/or system shown in FIGS. 1A and 1B may be implemented as multiple, distributed devices and/or systems. Additionally or alternatively, a set of devices and/or systems (e.g., one or more devices or systems) of environment 100 and/or procedure system 106 may perform one or more functions described as being performed by another set of devices and/or systems of environment 100 and/or procedure system 106.

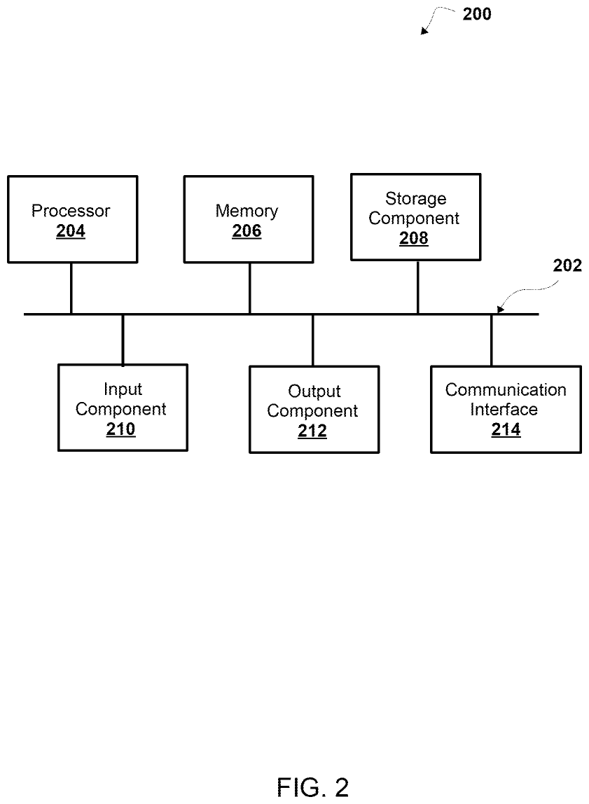

Referring now to FIG. 2, FIG. 2 is a diagram of example components of a device 200. Device 200 may correspond to one or more devices of imaging system 120, injection system 130, local system 140, support system 102, procedure system 106, database system 108 and/or communications network 110. In some non-limiting embodiments or aspects, one or more devices of imaging system 120, injection system 130, local system 140, support system 102, procedure system 106, database system 108 and/or communications network 110 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of non-transitory computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 (e.g., a central processing unit (CPU), a graphics processing unit (GPU), etc.) executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

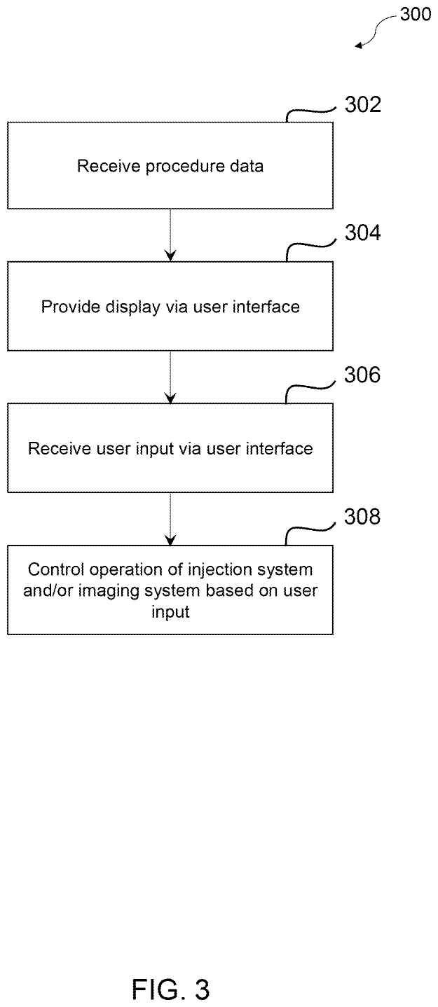

Referring now to FIG. 3, FIG. 3 is a flowchart of non-limiting embodiments or aspects of a process 300 for implementing a remote console for use in diagnostic imaging procedures. In some non-limiting embodiments or aspects, one or more of the steps of process 300 may be performed (e.g., completely, partially, etc.) by support system 102 (e.g., one or more devices of support system 102, etc.). In some non-limiting embodiments or aspects, one or more of the steps of process 300 may be performed (e.g., completely, partially, etc.) by another device or a group of devices separate from or including support system 102, such as procedure system 106 (e.g., one or more devices of procedure system 106, etc.), database system 108 (e.g., one or more devices of database system 108, etc.), imaging system 120 (e.g., one or more devices of imaging system 120, etc.), injection system 130 (e.g., one or more devices of injection system 130, etc.), and/or local system 140 (e.g., one or more devices of local system 140, etc.).

As shown in FIG. 3, at step 302, process 300 includes receiving procedure data. For example, support system 102 may receive procedure data. As an example, support system 102 may receive, from at least one of injection system 130 including injector 132 configured to deliver fluid to a patient and imaging system 120 including scanner 122 configured to scan the patient, procedure data associated with a procedure for the patient. In such an example, support system 102 may receive, from database system 108, the procedure data associated with the procedure for the patient.

Support system 102 may include support user interface 103, and/or at least one of injection system 130 and imaging system 120 may include at least one further user interface (e.g., scanner user interface 123 and/or injector user interface 133, etc.) different than support user interface 103. Support system 102 may be located at support location 101, and injection system 130 and imaging system 120 may be located at procedure location 104 different than support location. 101. For example, support user interface 103 may be located at a different location that is remote from procedure location 104 at which scanner user interface 123 and/or injector user interface 133 are located. As an example, support location 101 including support system 102 that includes support user interface 103 may include a remote console control room, an isolation room, a home office, and/or the like, and/or procedure location 104 including scanner 122, scanner user interface 123, injector 132, and injector user interface 133 may include at least one of the following locations: a hospital, an imaging center, a mobile imaging trailer, a vehicle, or any combination thereof. In such an example, injection system 130 may be in communication with the imaging system 120 (e.g., via a controller area network (CAN), etc.).

In some non-limiting embodiments or aspects, support user interface 103 and scanner user interface 123 and injector user interface 133 may enable an operator or local technologist (e.g., a radiographer, a radiology technologist, a medical assistant, etc.) at procedure location 104 to be in ongoing, constant, and/or as needed communication with a user or virtual imaging specialist (e.g., a radiologist, a fellow, an experienced radiology technologist, etc.) at support location 101 (e.g., via headset communicators, via chat windows on UI displays, etc.).

Procedure data may include at least one of the following: at least one image captured by an image capture device (e.g., a camera, etc.) associated with at least one of injection system 130 and imaging system 120; information received via scanner user interface 123 and/or injector user interface 133; sensor data captured by at least one sensor associated with at least one of injection system 130 and imaging system 120; user input received via support user interface 103; patient data; or any combination thereof. In some non-limiting embodiments or aspects, procedure data includes indirect user input including information and/or data automatically gathered by support system 102 during a user's use of injection system 130 including injector 132 and/or imaging system 120 including scanner 122, such as, usage metrics, user selections, and/or the like.

An image captured by an image capture device (e.g., a camera, etc.) associated with at least one of injection system 130 and imaging system 120 may include at least one of the following: one or more images of the patient; one or more images of an operator of at least one of injection system 130 and imaging system 120 (e.g., of injector 132 and/or scanner 122, etc.); one or more images of an IV access location on the patient; one or more images of injector 132; one or more images of scanner 122; or any combination thereof. For example, injection system 130, imaging system 120, and/or local system 140 may include an image capture device configured to monitor an environment surrounding injector 132 and/or scanner 122 (e.g., a scan room, a control room, etc.).

Information received via scanner user interface 123 and/or injector user interface 133 may include at least one of the following: an imaging prescription; a pre-procedure checklist; an indication of patient consent; an adverse reaction checklist; at least one of a selection, a programming, and a modification of an injection protocol; at least one of a selection, a programming, and a modification of an imaging protocol; a request to initiate the operation; a request to end the procedure; information captured at an end of the procedure; information for a secondary capture; information for a billing procedure; or any combination thereof.

Sensor data may include parameters associated with the patient, the scanner 122, the injector 132, an additional or auxiliary device, and/or the operator. For example, sensor data associated with the patient, scanner 122, injector 132, and/or the operator may include at least one of the following: a movement associated with the patient during the procedure; a sound associated with the patient (e.g., cry for help, etc.) during the procedure; a patient weight on scanner 122; a scan timing of scanner 122; a communication state between scanner 122 and injector 132; one or more images or scans of the patient captured by scanner 122; an amount of noise (e.g., a signal-to-noise ratio (SNR), etc.) associated with the one or more images or scans of the patient captured by scanner 122; artifacts (e.g., a level of artifacts, a number of artifacts, etc.) associated with the one or more images or scans of the patient captured by scanner 122; an enhancement or saturation level associated with the one or more images or scans of the patient captured by scanner 122; an imaging signal from a region of interest at a time or over a length of time; a maintenance status of scanner 122; a flow rate during one or more injections (e.g., a maximum, a minimum, an average, a total, etc.) by injector 132, an ECG gating sequence; a pulse of a patient; a flow rate programmed to be achieved for one or more injections by injector 132; a volume pumped and/or delivered during one or more injections by injector 132 (e.g., a maximum, a minimum, an average, a total, etc.); a volume programmed to be delivered during one or more injections by injector 132; a duration of time of one or more injections by injector 132 (e.g., a maximum, a minimum, an average, a total, etc.); a difference between the flow rate during the one or more injections and a programmed flow rate (e.g., set by an injection parameter of an injection protocol, etc.) of the one or more injections by injector 132; a difference between the volume pumped and/or delivered during the one or more injections and a programmed volume to be pumped and/or to be delivered (e.g., set by an injection parameter of an injection protocol, etc.) during the one or more injections by injector 132; a number of injections performed by injector 132; an achieved pressure of one or more injections (e.g., a maximum, a minimum, an average, a total, etc.) by injector 132; a difference between the achieved pressure of the one or more injections and a programmed pressure to be achieved (e.g., set by an injection parameter of an injection protocol, etc.) during the one or more injections by injector 132; a pressure limit or threshold beyond which the injection system is programmed to cease delivery of an injection by injector 132; a duration of time powered-on (e.g., a maximum, a minimum, an average, a total, etc.); a number of times power has been cycled for scanner 122 and/or injector 132; an energy consumption (e.g., a maximum, a minimum, an average, a total, etc.) of scanner 122 and/or injector 132; a linear amount of power delivered or used (e.g., an integral of ((pressure)*(flow rate))/(time), etc.) by scanner 122 and/or injector 132; a non-linear amount of power delivered or used (e.g., an integral of a f(pressure)*(time), etc.) by scanner 122 and/or injector 132; a voltage (e.g., a maximum, a minimum, an average, a total, etc.) at scanner 122 and/or injector 132; a resistance at scanner 122 and/or injector 132; a current at scanner 122 and/or injector 132; a noise or signal level at scanner 122 and/or injector 132; a mechanical force produced and/or the like (e.g., a maximum, a minimum, an average, a total, etc.) by a motor of injector 132; a number of camera reads; a number and/or a type of error codes received by scanner 122 and/or injector 132; a number, a duration and/or a type of user interface keys actuated (e.g., pressed, etc.) at scanner 122 and/or injector 132; power line condition associated with scanner 122 and/or injector 132; a temperature and/or a humidity within scanner 122 and/or injector 132 (e.g., a maximum, a minimum, an average, a total, etc.); a temperature and/or a humidity of an environment surrounding scanner 122 and/or injector 132 (e.g., a maximum, a minimum, an average, a total, etc.); a vibration frequency and/or amplitude (e.g., a maximum, a minimum, an average, a total, etc.) of scanner 122 and/or injector 132; a number of times scanner 122 and/or injector 132 is cleaned; a staff rating of wear (e.g., a numerical rating, etc.) for scanner 122 and/or injector 132; a staff rating of cleanliness (e.g., a numerical rating, etc.) for scanner 122 and/or injector 132; a service record of service (e.g., a number of services performed, a type of services performed, etc.) for scanner 122 and/or injector 132; a system rating of wear (e.g., a numerical rating, etc.) associated with scanner 122 and/or injector 132; a system rating of cleanliness (e.g., a numerical rating, etc.) associated with scanner 122 and/or injector 132; a number of one or more disposables (e.g., syringes, transfer sets, single-use, multi-use, etc.) sold to and/or used by an associated procedure location 104; an amount of contrast used by injector 132; a type of contrast used by injector 132; a vial size of contrast used by injector 132; a number of injectors at procedure location 104; a turnover rate of users or operators associated with procedure location 104; an identifier of a user or operator associated with one or more operations or uses of scanner 122 and/or injector 132; an identifier of a customer associated with procedure location 104; an indication of liquid within scanner 122 and/or injector 132 (e.g., as detected or measured by one or more liquid sensors, etc.); an amount (e.g., a maximum, a minimum, an average, a total, etc.) of x-ray radiation, RF exposure, magnetic field exposure, and/or the like in an environment surrounding scanner 122 and/or injector 132; one or more injection protocols used for one or more injections; one or more imaging protocols used for one or more scans; and/or the like.

In some non-limiting embodiments or aspects, sensor data associated with injector 132 includes one or more exemplary data types, information types, and/or parameters that are disclosed in U.S. patent application Ser. No. 10/143,562, filed on May 10, 2002, issued as U.S. Pat. No. 7,457,804; U.S. patent application Ser. No. 12/254,318, filed on Oct. 20, 2008, issued as U.S. Pat. No. 7,996,381; U.S. patent application Ser. No. 13/180,175, filed on Jul. 11, 2011, issued as U.S. Pat. No. 8,521,716; International Patent Application Publication No. WO2017/027724A1, published Feb. 16, 2017, which was filed as International Application No. PCT/US2016/046587 on Aug. 11, 2016; and International Patent Application Publication No. WO2017/040152A1, published Mar. 9, 2017, which was filed as International Application No. PCT/US2016/048441 on Aug. 24, 2016, the disclosures of each of which are incorporated herein by reference in their entireties.

In some non-limiting embodiments or aspects, sensor data associated with the patient may include at least one of the following parameters: firstly, parameters which may be affected by an injection and/or changes in patient wellbeing such as a heart rate; a sound or vibration (e.g., a sound or vibration associated with a fluid inflow, a sound or vibration proximate an injection site, etc.); a temperature (e.g., a temperature of a fluid inflow, a temperature proximate an injection site, a localized temperature, a tissue temperature, etc.); an oxygen saturation level (e.g., an oxygen saturation of a fluid inflow, an oxygen saturation proximate an injection site, etc.); a pulse rate; an ECG; a body fat/water-content ratio; a tissue impedance; a vessel distribution level; a vessel diameter; a hydration level; a hematocrit level; a skin resistivity; a blood pressure; a muscle tension level; a light absorptivity level; a shaking or trembling or a movement/motion (e.g., a yes, a no, a level, etc.); an arm position; an arm circumference; a respiration rate; a respiration depth; an amount of absorbed radiation; a tightness, a position stability, and/or a contact integrity of a contact sensor device; an amount of swelling and/or displacement; an EMG; a skin color; a surface vessel dilation (flushing) amount; a bio-impedance; a light absorptivity; an inflammation level; secondly, parameters which are not likely to be immediately affected by an injection and/or changes in a patient wellbeing such as a fat/muscle ratio; a hemoglobin level; and thirdly, environmental parameters such as an environmental temperature of an environment surrounding the patient, a barometric pressure in an environment surrounding the patient; an ambient light level; an ambient sound level; or any combination thereof.

In some non-limiting embodiments or aspects, support system 102 may determine sensor data to compare expected performance among contrast media using a concentration/viscosity ratio as described by McDermott M, Kemper C, Barone W, Jost G, Endrikat J. Impact of CT Injector Technology and Contrast Media Viscosity on Vascular Enhancement: Evaluation in a Circulation Phantom. Br J Radiol. 2020 May 1; 93 (1109): 20190868. doi: 10.1259/bjr.20190868. Epub 2020 Feb. 20. PMID: 32017607; PMCID: PMC7217576, the disclosure of which is incorporated herein by reference in its entirety.

In some non-limiting embodiments or aspects, procedure data may include at least one of the following conditions determined based on sensor data: an overpressure disarming of injector 132; a premature injection termination by injector 132; an unexpected injection timing associated with one or more fluid injections by injector 132; an unexpected scanner timing associated with one or more scans by scanner 122; an indication that no injection occurred when an injection was expected; an injection flow rate that violates a threshold flow rate; an injection volume that violates a threshold volume; an injection pressure that violates a threshold pressure; a violation of a catheter rule set; a violation of pediatric injector guardrails; a communication interface drop out or error at scanner 122 and/or injector 132; an amount of noise (e.g., a signal-to-noise ratio (SNR), etc.) associated with the one or more images or scans of the patient captured by scanner 122 that violates a threshold noise level; an occurrence of artifacts (e.g., a level of artifacts, a number of artifacts, etc.) associated with the one or more images or scans of the patient captured by scanner 122; an enhancement or saturation level associated with the one or more images or scans of the patient captured by scanner 122 that violates a threshold enhancement or saturation level; a venous contamination; a fluid path leak or break indicated and/or associated with a pressure curve; a premature enhancement; a patient movement; a patient adverse reaction; a maintenance status associated with scanner 122 and/or injector 132; an inexperienced operator (e.g., operator hesitancy, programming statistics, etc.); an actuation of a help button; an extravasation associated with the one or more injections; an air injection associated with the one or more injections; an occlusion associated with the one or more injections; a patency of an IV line; an impedance of injector 132 that violates a threshold impedance (e.g., to predict pressure limiting, etc.); or any combination thereof.

In some non-limiting embodiments or aspects, sensor data is captured by the at least one sensor during at least one of the following: a setup of injector 132 and/or scanner 122; an arming of injector 132; a period of time when injector 132 is armed; a keep vein open (KVO) operation by injector 132; a delivery of a test bolus and/or a tracking bolus to the patient by injector 132; a delivery of a contrast media to the patient by injector 132; a period of time after delivery of the contrast media to the patient; a delivery of a saline flush to the patient by the injector; a period of time after delivery of the saline flush to the patient; a scanning of the patient by the scanner; or any combination thereof.

In some non-limiting embodiments or aspects, procedure data includes patient data. Patient data may include at least one of the following parameters associated with a patient: an age; a gender; a weight; a height; a body mass index (BMI); a cardiac output; a type of procedure to be performed; a prior chemotherapy status, such as an adverse peripheral venous status due to long-term oncological treatment, and/or the like (e.g., a yes, a no, a number of cycles of chemotherapy received, etc.); an estimated glomerular filtration rate (eGFR) (e.g., an eGFR of less than 45 ml/min/1.73 m², etc.); a prior injection history including one or more allergic reactions to contrast media; a thyroid stimulating hormone (TSH) level; a Triiodothyronine (FT3) Thyroxine (FT4) ratio (FT3/FT4); an amount or level of an environmental influence (e.g., a regional iodine saturation of nutrition amount or level for a region or location associated with the patient, etc.); a prior reaction to a previous contrast media injection status (e.g., a yes, a no, a level, etc.); an atopic disorder status (e.g., a yes, a no, a level, etc.); a medical status as it relates to existence of diabetes and/or hypertension, such as, a diabetic nephropathy status, and/or the like (e.g., a yes, a no, a level, etc.); a congestive heart failure status (e.g., a yes, a no, a level, etc.); a hematocrit level; a known or suspected renal failure status (e.g., a yes, a no, a level, etc.); a malignancy status (e.g., a yes, a no, a level, etc.); an implanted device for a central venous access status (e.g., a yes, a no, a level, etc.); a type of a medication; a type of contrast media to be administered in a contrast media injection; a type of a contrast media injection and/or imaging exam; a flow rate associated with a contrast media injection; a catheter gauge associated with a contrast media injection; a total volume of fluid associated with a contrast media injection; a pressure curve associated with a contrast media injection, an injection site associated with a contrast media injection; a location of a patient IV access point; a quality of the patient IV access point (e.g., defined by an expertise level of a technician associated with placing the IV access point, etc.); or any combination thereof. In some non-limiting embodiments or aspects, patient data may include sensor data determined before a contrast media injection is administered to a patient and/or sensor data determined during and/or after one or more previous contrast media injections previously administered to the patient.

In some non-limiting embodiments or aspects, support system 102 retrieves information associated with at least one of the procedure and the patient (e.g., procedure data, patient data, etc.) from one or more database systems 108. For example, support system 102 may retrieve information associated with at least one of the procedure and the patient from at least one of the following: an electronic medical record (EMR) system; a radiology information system (RIS); a picture archiving and communication system (PACS); a hospital information system (HIS); a hospital enterprise information system; a cross location network; or any combination thereof.

As shown in FIG. 3, at step 304, process 300 includes providing a display via a user interface. For example, support system 102 may provide a display via a user interface. As an example, support system 102 may provide, via user interface 103, a display. In such an example, the display may be generated based on the procedure data.

In some non-limiting embodiments or aspects, a display provided via support user interface 103 may include an alert. For example, injection system 130 and/or imaging system 120 may generate the alert based on at least one of the following: procedure data; patient data; sensor data; user input received via support user interface 103; user input received via scanner user interface 123 and/or injector user interface 133; or any combination thereof. As an example, an alert may be associated with one or more of the parameters or conditions described herein as determined based on the procedure data, the patient data, the sensor data, the user input received via support user interface 103, and/or the user input received via scanner user interface 123 and/or injector user interface 133. In such an example, the display may include an alert, and/or injection system 130 and/or imaging system 120 may generate the alert based on patient data before arming injector 132 for the procedure.

An alert provided via support user interface 103 may include at least one of the following: an indication that the procedure is complete; an indication of whether the procedure executed correctly; a sound emitted by scanner 122 that is replicated by support system 102; an indication that an operator appears hesitant during programming; an indication of whether an operator is viewing a correct injector at a correct procedure location with a correct patient; a real-time display of injection parameters (e.g., a pressure graph, injection phases, etc.); an indication of an adverse event (e.g., an extravasation, etc.); a selected injection protocol; a selected imaging protocol; an indication of a quality of one or more images of the patient captured by scanner 122; or any combination thereof.

In some non-limiting embodiments or aspects, a display provided via support user interface 103 may mirror a display provided via scanner user interface 123 and/or injector user interface 133. In some non-limiting embodiments or aspects, support user interface 103, scanner user interface 123, and/or injector user interface 133 may include independent workflow aware user interfaces as described by U.S. Pat. No. 11,090,440, published Aug. 17, 2021, the disclosure of which is incorporated by reference in its entirety.

In some non-limiting embodiments or aspects, a display provided via support user interface 103 includes a prompt to confirm or approve an operation of scanner 122 and/or injector 132. For example, an operation may include at least one of the following operations: changing a display of scanner user interface 123 and/or injector user interface 133; receiving further user input via scanner user interface 123 and/or injector user interface 133; setting an injection protocol at injector 132; setting an imaging protocol at scanner 122; initiating a loading process with injector 132; initiating a data capture with the injector 132; arming injector 132; initiating a keep vein open (KVO) procedure with injector 132; initiating a saline test injection with injector 132; initiating a drug injection with injector 132; initiating a contrast media test injection with injector 132; initiating a contrast media injection with injector 132; initiating a scan with scanner 122; aborting the test injection with injector 132; aborting the contrast media injection with injector 132; aborting the scan with scanner 122; initiating a saline flush injection with the injector; aborting the saline flush injection with the injector; ending the procedure for the patient; or any combination thereof. As an example, an operation and/or a display associated therewith may include one or more of the operations described by U.S. Pat. No. 11,232,862, published Jan. 25, 2022, the disclosure of which is incorporated by reference in its entirety, and which may be used for predictive maintenance of injection systems. In some non-limiting embodiments or aspects, a contrast media injection may include a dual flow injection in which a controlled ratio of each of contrast media and saline are delivered together, either simultaneously or sequentially in small enough boluses that the contrast media and the saline effectively mix before being imaged.

In some non-limiting embodiments or aspects, an operation of injector 132 may include delivering a test bolus and/or determining an injection protocol based thereon as described in International Patent Application Publication No. WO2020247370A1, published Dec. 10, 2020, which was filed as International Patent Application No. PCT/US2020/035699 on Jun. 2, 2020, the disclosure of which is incorporated by reference in its entirety.

In some non-limiting embodiments or aspects, procedure data includes one or more images (and/or one or more segments thereof) of the patient captured by scanner 122, and/or support system 102 (and/or imaging system 120, etc.) processes the one or more images to select a subset of the one or more images, and/or support system 102 generates the display based on the selected subset of the one or more images. For example, support system 102 (and/or imaging system 120, etc.) may process the one or more images using a machine learning model and/or other image processing algorithms to select the subset of the one or more images according to one or more quality controls or thresholds.

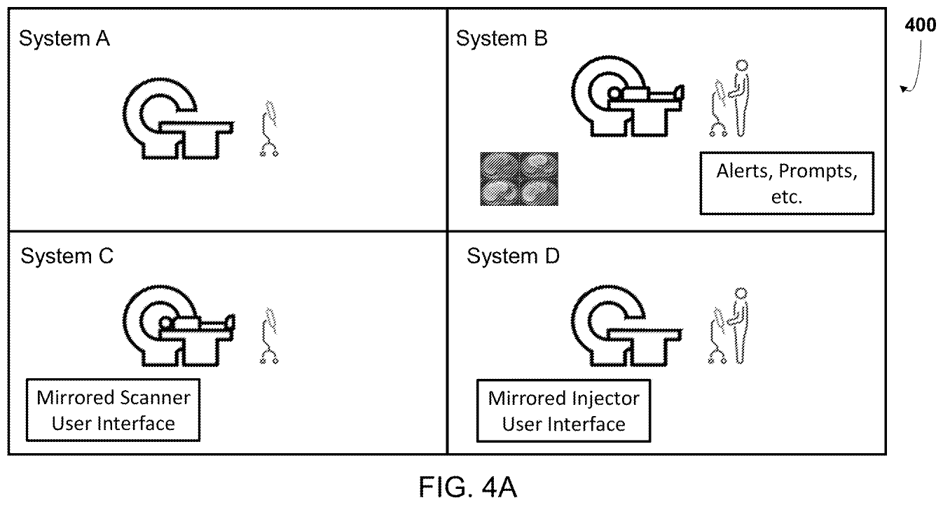

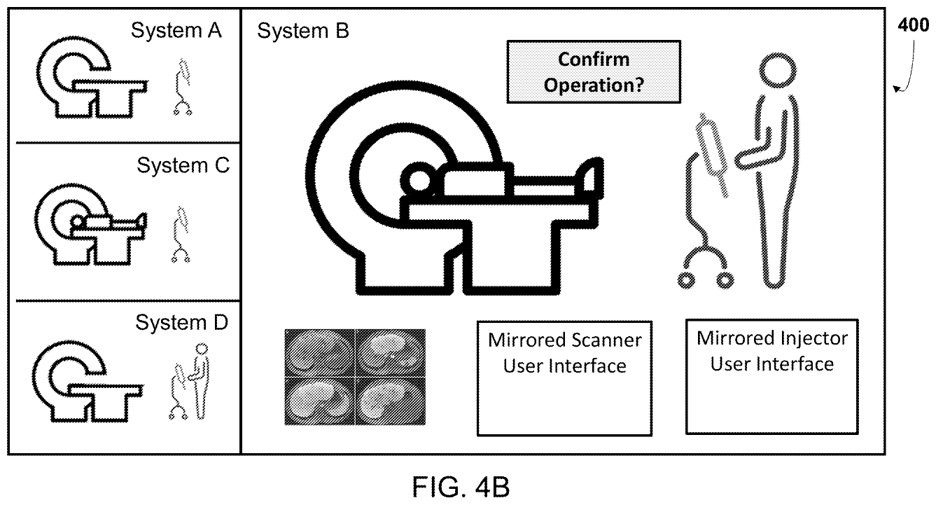

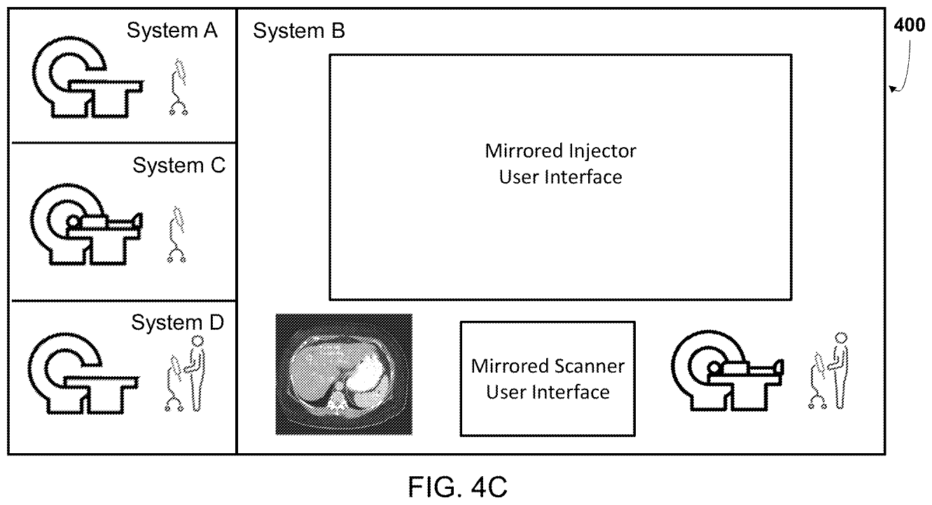

Referring also to FIGS. 4A-4C, FIGS. 4A-4C illustrate an implementation 400 of non-limiting embodiments or aspects of a user interface of a remote console for use in diagnostic imaging procedures. As shown in FIG. 4A, support system 102 may provide, via user interface 103, a display. For example, the display may include a plurality of segmented displays or sections corresponding to a plurality of procedure systems 106 (e.g., System A, System B, System C, System D, etc.). As an example, the segmented display or section corresponding to each procedure location 106 may be generated based on the procedure data associated with and/or received from that procedure system 106. For example, as shown in FIG. 4A, the segmented display or section for each procedure system 106 may include one or more of: a real-time display or live video feed of each location, which may be in various procedure states; one or more alerts associated with each location; a mirrored scanner user interface associated with each location; a mirrored injector user interface associated with each location; and/or the like.

As further shown in FIG. 4A, the segmented display or section for each procedure system 106 may be a same or similar size as each other section. For example, support system 102 may provide, via user interface 103, the display in a manner that does not overtly draw the attention of a virtual imaging specialist monitoring the plurality of procedure systems 106 to one procedure system 106 over the other procedure systems. However, support system 102 may automatically provide, via user interface 103, based on the procedure data, the display with one or more of the segmented displays or sections for one or more of the procedure systems 106 in an enlarged or zoomed in manner as compared to the other segmented displays or sections for the other procedure systems. For example, as shown in FIG. 4B, support system 102 may automatically provide, via user interface 103, based on the procedure data, the display with the segmented display or section for System B in an enlarged, zoomed in, and/or highlighted manner as compared to the other segmented displays or sections for the other procedure systems. As an example, support system 102 may automatically provide, via user interface 103, the display with the segmented display or section for System B in an enlarged, zoomed in, and/or highlighted manner in response to at least one of: generating or receiving an alert associated with System B, generating or receiving a prompt to confirm or approve an operation of scanner 122 and/or injector 132 of System B, receiving user input via user interface 103, receiving user input via scanner user interface 123 and/or injector user interface 133, any combination thereof, and/or the like. For example, support system 102 may be configured to automatically draw the attention of the virtual imaging specialist monitoring the plurality of procedure systems 106 to a procedure system 106 or operator thereof that is currently and/or most urgently requesting or requiring input and/or help from the virtual imaging specialist, such as for the virtual imaging specialist to confirm an operation of imaging system 120 and/or injection system 130 before initiation thereof, and/or like.

Support system 102 may automatically provide, via user interface 103, based on the procedure data, within one or more of the segmented displays or sections for one or more of the procedure systems 106 a portion thereof in an enlarged or zoomed in manner as compared to other portions of that segmented display or section. For example, as shown in FIGS. 4B and 4C, support system 102 may automatically provide, via user interface 103, based on the procedure data, within the segmented display or section for System B, a portion thereof in an enlarged, zoomed in, and/or highlighted manner as compared to the other portions thereof. As an example, support system 102 may automatically provide, via user interface 103, a portion of the segmented display or section for System B in an enlarged, zoomed in, and/or highlighted manner in response to at least one of: generating or receiving an alert associated with System B, generating or receiving a prompt to confirm or approve an operation of scanner 122 and/or injector 132 of System B, receiving user input via user interface 103, receiving user input via scanner user interface 123 and/or injector user interface 133, any combination thereof, and/or the like. For example, support system 102 may be configured to automatically draw the attention of the virtual imaging specialist monitoring System B to information and/or data associated with a current alert and/or prompt to be addressed at System B, such as for the virtual imaging specialist to confirm an operation of imaging system 120 and/or injection system 130 before initiation thereof as shown in FIG. 4B, for the virtual imaging specialist to monitor the operation of imaging system 120 and/or injection system 130 during execution thereof as shown in FIG. 4C, and/or like.

As shown in FIG. 3, at step 306, process 300 includes receiving user input via a user interface. For example, support system 102 may receive user input via a user interface. As an example, support system 102 may receive, via support user interface 103, user input.

In some non-limiting embodiments or aspects, user input includes at least one of a selection, a programming, and a modification of at least one of an injection protocol and an imaging protocol. For example, support system 102 and/or injection system 130 may adjust, based on user input, at least one of a maximum flow rate, a maximum pressure, an injection duration, a total volume of fluid, or any combination thereof, of a fluid injection.

In some non-limiting embodiments or aspects, user input includes a response (e.g., a confirmation, a denial, etc.) to a prompt to confirm or approve an operation of scanner 122 and/or injector 132. For example, if support user interface 103 includes a display that includes a prompt to confirm an operation of imaging system 120 and/or injection system 130, support system 102 may receive, via support user interface 103 from a user, a response that confirms or denies the operation.

In some non-limiting embodiments or aspects, user input includes an instruction to scanner 122 and/or injector 132 to initiate, modify, and/or perform an operation of scanner 122 and/or injector 132. For example, user input may include an instruction to scanner 122 and/or injector 132 to initiate, modify, and/or perform at least one of the following operations: changing a display of scanner user interface 123 and/or injector user interface 133; receiving further user input via scanner user interface 123 and/or injector user interface 133; setting an injection protocol at injector 132 (e.g., phases, volume, flow rate, pressure limits, fluid type, etc.); setting an imaging protocol at scanner 122; initiating a loading process with injector 132; initiating a data capture with the injector 132; arming injector 132; initiating a keep vein open (KVO) procedure with injector 132; initiating a saline test injection with injector 132; initiating a drug injection with injector 132; initiating a contrast media test injection with injector 132; initiating a contrast media injection with injector 132; initiating a scan with scanner 122; aborting the test injection with injector 132; aborting the contrast media injection with injector 132; aborting the scan with scanner 122; initiating a saline flush injection with the injector; aborting the saline flush injection with the injector; ending the procedure for the patient; or any combination thereof. As an example, an operation and/or user input associated therewith may include one or more of the operations described by U.S. Pat. No. 11,232,862, published Jan. 25, 2022, the disclosure of which is incorporated by reference in its entirety.

In some non-limiting embodiments or aspects, user input includes injection protocol parameters (e.g., phases, volumes, flow rates, pressure limits etc.); a type of fluid associated with a fluid delivery (e.g., a contrast type, saline, etc.); quality assurance guardrail parameters; patient health information (e.g., a CIN risk, allergies, etc.); an air check confirmation; a checkbox for patient characteristics to guide protocol selection; one or more safety inputs, checks, and/or halts; documentation of an adverse event; a location and/or a method of IV access; or any combination thereof. In some non-limiting embodiments or aspects, user input includes system enabled communications between a first user at scanner 122 and a second user at support system 102 (e.g., at a remote console, etc.) such as, for example, voice communications, voice capture and camera capture of the scanner space or console (optionally with the user visible while working), video conferencing communications, text communications, and/or the like.

In some non-limiting embodiments or aspects, user input may include any of the user input described by International Patent Application Publication No. WO2021222771A1, published Nov. 4, 2021, which was filed as International Patent Application No. PCT/US2021/030210 on Apr. 30, 2021, the entire contents of which is hereby incorporated by reference.

As shown in FIG. 3, at step 308, process 300 includes controlling an operation of an injection system and/or an imaging system based on user input. For example, support system 102 may control an operation of an injection system and/or an imaging system based on user input. As an example, support system 102 may control, based on the user input, an operation of at least one of the injection system and the imaging system. In such an example, support system 102 may control injection system 130 and/or imaging system 120 to at least one of modify and abort the operation. As an example, support system 102 may control injection system 130 to modify or abort an operation that includes delivering fluid to the patient with injector 132. As an example, support system 102 may control imaging system 120 to modify or abort an operation that includes scanning the patient with scanner 122.

In some non-limiting embodiments or aspects, support system 102 may control, based on the user input, scanner 122 and/or injector 132 to initiate and/or perform at least one of the following operations: changing a display of scanner user interface 123 and/or injector user interface 133; receiving further user input via scanner user interface 123 and/or injector user interface 133; setting an injection protocol at injector 132; setting an imaging protocol at scanner 122; initiating a loading process with injector 132; initiating a data capture with the injector 132; arming injector 132; initiating a keep vein open (KVO) procedure with injector 132; initiating a saline test injection with injector 132; initiating a drug injection with injector 132; initiating a contrast media test injection with injector 132; initiating a contrast media injection with injector 132; initiating a scan with scanner 122; aborting the test injection with injector 132; aborting the contrast media injection with injector 132; aborting the scan with scanner 122; initiating a saline flush injection with the injector; aborting the saline flush injection with the injector; ending the procedure for the patient; or any combination thereof.

In some non-limiting embodiments or aspects, if a display of support user interface 103 includes a prompt to confirm an operation of injection system 130 and/or imaging system 120, support system 102 may control the operation of injection system 130 and/or imaging system 120 by preventing injection system 130 and/or imaging system 120 from performing the operation until a response to the prompt is received via support user interface 103.

In some non-limiting embodiments or aspects, support system 102 receives, from the injection system 130 and/or imaging system 120 via injection user interface 133 and/or imaging user interface 123, authentication data (e.g., a username, a password, etc.) associated with a user account (e.g., a user account of an operator, etc.). For example, support system 102 may restrict, based on an authorization level associated with the user account, one or more operations of injection system 130 and/or imaging system 120 from being initiated and/or modified via injection user interface 133 and/or imaging user interface 123 by the user account. As an example, support system 102 may determine the authorization level associated with the user account on the authentication data. In such an example, the authorization level associated with the user account may be selected from one of the following authorization levels: a first authorization level at which the user account is restricted from modifying at least one of an injection protocol and a scanning protocol; a second authorization level at which the user account is restricted from modifying a first subset of parameters of the at least one of the injection protocol and the scanning protocol and enabled to modify a second subset of parameters of the at least one of the injection protocol and the scanning protocol; and a third authorization level at which the user account is enabled to modify each parameter of the at least one of the injection protocol and the scanning protocol.

In some non-limiting embodiments or aspects, support system 102 may further determine an authorization level associated with a user account based on at least one of the following: a number of procedures associated with the user account; a quality, experience, and/or other rating associated with the user account; a type and/or an aspect of the procedure; a type of the patient; or any combination thereof.

In some non-limiting embodiments or aspects, procedure system 106 may include a fail-safe that is to fail in a mode such that procedure system 106 is still locally operational to take images and/or inject contrast (e.g., imaging system 120 and injection system 132 are still locally operational, etc.) even if communications with support system 102 fails and/or some other aspect of the support system 102 fails.

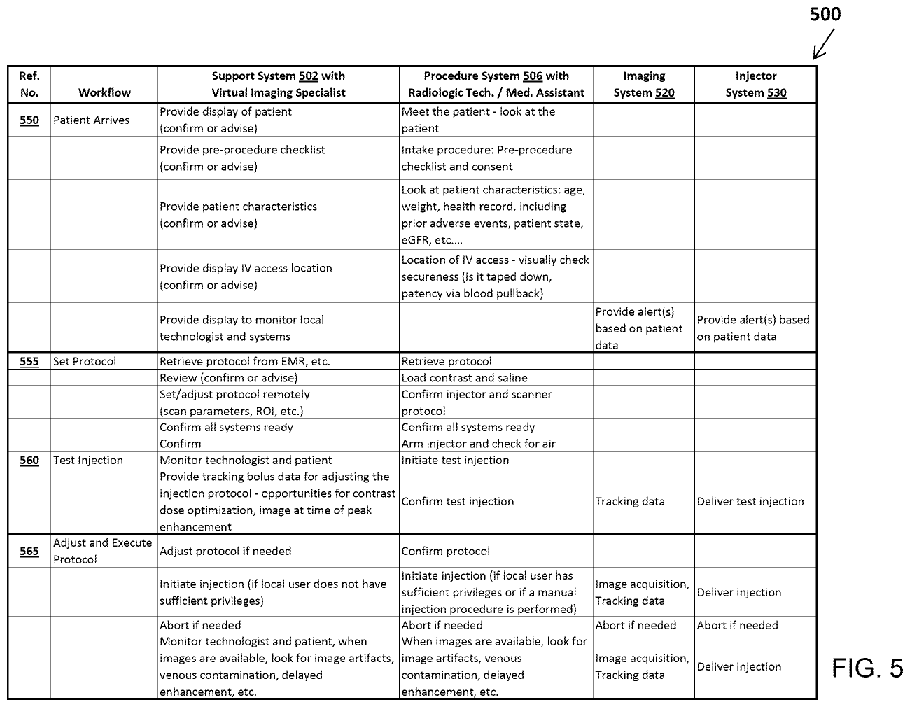

Referring now to FIG. 5, FIG. 5 is a diagram of an implementation 500 of non-limiting embodiments or aspects of a process for implementing a remote console for use in diagnostic imaging procedures. As shown in FIG. 5, implementation 500 includes support system 502, procedure system 506, imaging system 520, and/or injection system 530. In some non-limiting embodiments or aspects, support system 502 can be the same as or similar to support system 102. In some non-limiting embodiments or aspects, procedure system 506 can be the same as or similar to procedure system 106. In some non-limiting embodiments or aspects, imaging system 520 can be the same as or similar to imaging system 120. In some non-limiting embodiments or aspects, injection system 530 can be the same as or similar to injection system 130.

As shown by reference number 550 in FIG. 5, support system 502 may provide a display of the patient to the virtual imaging specialist (e.g., the remote user) (e.g., in response to a patient arriving at a location of procedure system 506, etc.). For example, a radiologic technologist (and/or medical assistant, the local user, etc.) at the location of procedure system 506 may meet the patient and confirm the identity of the patient, and the virtual imaging specialist may concurrently confirm the identity of the patient and/or advise the radiologic technologist via support system 502. A prescription for the procedure (e.g., a CT liver procedure/study, a MR cardiac procedure/study, etc.), the pre-procedure checklist, and/or patient characteristics may be obtained by procedure system 506 and/or input or imported thereto by the radiologic technologist from an EHR and/or EMR system. As further shown by reference number 550, support system 502 may provide the procedure checklist, patient characteristics, IV access location, and/or the like to the virtual imaging specialist for the virtual imaging specialist to confirm the procedure checklist, the patient characteristics, the IV access location and/or advise the radiologic technologist with regard thereto. As still further shown by reference number 550, support system 502 may provide a display to the virtual imaging specialist to monitor the local radiologic technologist and/or systems (e.g., imaging system 520, injection system 530, etc.) for correct actions and/or alerts or potential adverse events generated based on the patient characteristics and/or pre-procedure checklist.

As shown by reference number 555 in FIG. 5, support system 502 and/or procedure system 506 may retrieve an injection and/or imaging protocol (e.g., from the EMR, from a protocol database, etc.). As further shown by reference number 555, support system 502 may provide the injection and/or imaging protocol to the virtual imaging specialist for the virtual imaging specialist to confirm the injection and/or imaging protocol and/or advise the radiologic technologist with regard thereto. The radiologic technologist may load the injector, which may be remotely monitored by the virtual imaging specialist via support system 502. As further shown by reference number 555, the virtual imaging specialist may remotely set, adjust, and/or confirm the injection and/or imaging protocol (e.g., scan parameters, a region of interest (ROI, etc.) via support system 502, and the radiologic technologist may arm the injector and check for air. Depending upon an authorization level of an on-site radiologic technologist, the technologist may do some, most, or all of the functions listed for the virtual imaging specialist, with the virtual imaging specialist, for example, being on call as requested by the on-site radiologic technologist and/or intervening when alerted by the support system 102, as well as monitoring the process in the procedure location and interacting as desired.

Non-limiting embodiments or aspects of authorization levels described herein are exemplary. For example, multi-level and/or multi-dimensional authorization schema with various authorizations may be used. As an example, a user may be able to use protocols: only as is, to modify a protocol, create a new protocol, to eliminate a protocol, and/or to override/block an action of another user.

As shown by reference number 560 in FIG. 5, the local radiologic technologist may initiate a test injection, and support system 502 may provide information or data to the virtual imaging specialist for monitoring the technologist and/or patient during the test injection. If the test injection includes only a saline injection, local sensors, the onsite technologist, and/or the virtual imaging specialist may confirm that the fluid flowed into the patient properly and/or that there was no extravasation and/or adverse event. If the test injection includes a contrast media used to capture information about the patient, the same monitoring for proper flow may occur as mentioned above and, as injection system 530 delivers the test injection, imaging system 520 may generate tracking bolus data for adjusting the injection protocol (e.g., contrast dose optimization, image at time of peak enhancement, etc.), which support system 502 may provide to the virtual imaging specialist. Example adjustments are described in International Patent Application Publication No. WO2020247370A1, published Dec. 10, 2020, which was filed as International Patent Application No. PCT/US2020/035699 on Jun. 2, 2020, the entire contents of which is hereby incorporated by reference.

As shown by reference number 565 in FIG. 5, after the test injection, the virtual imaging specialist may adjust the injection and/or imaging protocol via support system 502, which may be confirmed by the local radiologic technologist. As further shown by reference number 565 in FIG. 5, the virtual imaging specialist may initiate the injection via support system 502, for example, if the local radiologic technologist does not have sufficient system privileges to locally initiate the injection at procedure system 506, and/or the local radiologic technologist may initiate the injection via procedure system 506 if the local radiologic technologist has sufficient system privileges to locally initiate the injection at procedure system 506 and/or if a manual injection procedure is performed. At any time during the injection, the virtual imaging specialist may abort the procedure via support system 502 and/or support system 502 may automatically abort the procedure based on the procedure data, which may cause injection system 530 to stop the injection and/or imaging system 520 to stop imaging. At any time during the injection, imaging system 520 and/or injection system 530 may detect a condition and abort the injection, the imaging procedure, or a combination thereof. As still further shown by reference number 565 in FIG. 5, when images are available from imaging system 520, support system 502 may provide the images to the virtual imaging specialist for review, such as to identify image artifacts, venous contamination, delayed enhancement, and/or the like. If there are any problems determined from the review, support system 502 and/or the virtual imaging specialist may decide to repeat some or all of the imaging procedure.

After the procedure is completed, the virtual imaging specialist may assess the patient state and procedure quality via support system 502, which may archive or store the information and/or data associated with the procedure (e.g., in a database, etc.).

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A system, comprising:
a user interface; and
at least one processor programmed and/or configured to:
receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system;
provide, via the user interface, a display, wherein the display is generated based on the procedure data;
receive, via the user interface, user input; and
control, based on the user input, an operation of at least one of the injection system and the imaging system.

2. The system of claim 1, wherein the procedure data includes at least one of the following:
at least one image captured by a camera associated with at least one of the injection system and the imaging system;
information received via the at least one further user interface;
sensor data captured by at least one sensor associated with at least one of the injection system and the imaging system;
or any combination thereof.

3. The system of claim 2, wherein the procedure data includes the at least one image, and wherein the at least one image includes at least one of the following:
one or more images of the patient;
one or more images of an operator of at least one of the injection system and the imaging system;
one or more images of an IV access location on the patient;
one or more images of the injector;
one or more images of the scanner;
or any combination thereof.

4. The system of claim 2, wherein the procedure data includes the information received via the at least one further user interface, and wherein the information received via the at least one further user interface includes at least one of the following:
an imaging prescription;
a pre-procedure checklist;
an indication of patient consent;
an adverse reaction checklist;
at least one of a selection, a programming, and a modification of an injection protocol;
at least one of a selection, a programming, and a modification of an imaging protocol;
a request to initiate the operation;
a request to end the procedure;
information captured at an end of the procedure;
information for a secondary capture;
information for a billing procedure;
or any combination thereof.

5. The system of claim 2, wherein the procedure data includes the sensor data captured by the at least one sensor associated with the at least one of the injection system and the imaging system, and wherein the at least one sensor includes at least one of the following sensors:
a flow sensor;
a temperature sensor;
an accelerometer;
a vibration sensor;
a strain gauge;
a motor current sensor;
an optical sensor;
the scanner;
or any combination thereof, and
wherein the procedure data includes at least one of the following parameters determined based on the sensor data: a movement associated with the patient; a sound associated with the patient; a communication state between the scanner and the injector; one or more images of the patient captured by the scanner;
an amount of noise associated with the one or more image of the patient captured by scanner; an enhancement or saturation level associated with the one or more images of the patient captured by scanner; a flow rate during one or more fluid injections by the injector; a duration of the one or more fluid injections by the injector; a volume pumped and/or delivered during the one or more injections by the injector; an achieved pressure of the one or more injections by the injector; an imaging signal from a region of interest at a time or over a length of time; an ECG gating sequence; a pulse of a patient; or any combination thereof.

6. The system of claim 5, wherein the sensor data is captured by the at least one sensor during at least one of the following:
a setup of at least one of the injector and the scanner;
an arming of the injector;
a period of time when the injector is armed;
a keep vein open (KVO) operation by the injector;
a delivery of a test bolus and/or a tracking bolus to the patient by the injector;
a delivery of a contrast media to the patient by the injector;
a period of time after delivery of the contrast media to the patient;
a delivery of a saline flush to the patient by the injector;
a period of time after delivery of the saline flush to the patient;
a scanning of the patient by the scanner;
or any combination thereof.

7. The system of claim 5, wherein the display includes an alert, and wherein at least one of the injection system and the imaging system generates the alert based on at least one of the following:
the sensor data;
the user input;
patient data associated with the patient;
or any combination thereof.

8. The system of claim 1, wherein the procedure data includes patient data including at least one of the following parameters associated with the patient:
an age;
a weight;
a height;
a body mass index;
a cardiac output;
a risk or probability of an adverse event;
an adverse event;
an estimated glomerular filtration rate (eGFR);
a prior injection history including one or more allergic reactions to contrast media;
a location of a patient IV access point;
a quality of the patient IV access point;
or any combination thereof.

9. The system of claim 8, wherein the display includes an alert, and wherein at least one of the injection system and the imaging system generates the alert based on the patient data before arming the injector for the procedure.

10. The system of claim 1, wherein the at least one processor is further programmed and/or configured to retrieve information associated with at least one of the procedure and the patient from at least one of the following:
an electronic medical record (EMR) system;
a radiology information system (RIS);
a picture archiving and communication system (PACS);
a hospital information system (HIS);
a hospital enterprise information system;
a cross location network;
or any combination thereof.

11. The system of claim 1, wherein the user input includes at least one of a selection, a programming, and a modification of at least one of an injection protocol and an imaging protocol.

12. The system of claim 1, wherein the display includes a prompt to confirm the operation of the at least one of the injection system and the imaging system, and wherein the at least one processor is programmed and/or configured to control the operation of the at least one of the injection system and the imaging system by:
controlling the at least one of the injection system and the imaging system such that the at least one of the injection system and the imaging system is prevented from performing the operation until a response to the prompt is received via the user interface.

13. The system of claim 12, wherein the operation includes at least one of the following operations:
changing a display of the at least one further user interface;
receiving further user input via the at least one further user interface,
setting an injection protocol at the injector;
setting an imaging protocol at the scanner;
initiating a loading process with the injector;
initiating a data capture with the injector;
arming the injector;
initiating a keep vein open (KVO) procedure with the injector;
initiating a saline test injection with the injector;
initiating a drug injection with the injector;
initiating a contrast media test injection with the injector;
initiating a contrast media injection with the injector;
initiating a scan with the scanner;
aborting the saline test injection with the injector;
aborting the contrast media test injection with the injector;
aborting the contrast media injection with the injector;
initiating a saline flush injection with the injector;
aborting the saline flush injection with the injector;
aborting the scan with the scanner;
ending the procedure for the patient;
or any combination thereof.

14. The system of claim 1, wherein the operation includes at least one of delivering fluid to the patient with the injector and scanning the patient with the scanner, and wherein the at least one processor is programmed and/or configured to control the operation of the at least one of the injection system and the imaging system by:
controlling the at least one of the injection system and the imaging system such that the at least one of the injection system and the imaging system at least one of modifies and aborts the operation.

15. The system of claim 1, wherein the procedure data includes one or more images of the patient captured by the scanner, and wherein the at least one processor is programmed and/or configured to process the one or more images to select a subset of the one or more images, and wherein the display is generated based on the selected subset of the one or more images.

16. The system of claim 1, wherein the scanner includes at least one of the following: a magnetic resonance (MR) scanner; a computed tomography (CT) scanner; a positron emission tomography/computed tomography (PET/CT) scanner; a positron emission tomography/magnetic resonance (PET/MR) scanner; a single-photon emission computerized tomography (SPECT) scanner; an ultrasound system, an infrared imaging system, a photo-acoustic imaging system, or any combination thereof.

17. The system of claim 1, wherein the injection system, the imaging system, and the at least one further user interface are located at a procedure location, and wherein the user interface is located at a support location different than the procedure location.

18. The system of claim 17, wherein the procedure location includes at least one of the following locations: a hospital, an imaging center, a mobile imaging trailer, a vehicle, or any combination thereof.

19. The system of claim 1, wherein the user input includes at least one of the following:
a response to a prompt to confirm the operation of the at least one of the injection system and the imaging system;
an instruction to the at least one of the injection system and the imaging system to initiate, modify, and/or perform the operation of the at least one of the injection system and the imaging system;
or any combination thereof.

20. The system of claim 1, wherein the display includes at least one alert, and wherein the at least one alert includes at least one of the following: an indication that the procedure is complete; an indication of whether the procedure executed correctly; an indication that an operator appears hesitant during programming; a real-time display of measured parameters associated with a fluid injection; an indication of a detected adverse event; an indication of a quality of one or more images of the patient captured by the scanner; or any combination thereof.

21. The system of claim 1, wherein the at least one processor is programmed and/or configured to:
receive, from the at least one of the injection system and the imaging system via the at least one further user interface, authentication data associated with a user account;
restrict, based on an authorization level associated with the user account, one or more operations of at least one of the injection system and the imaging system from being initiated and/or modified via the at least one further user interface by the user account, wherein the authorization level associated with the user account is determined based on the authentication data.

22. The system of claim 21, wherein the authorization level is further determined based on at least one of the following:
a number of procedures associated with the user account;
a quality, experience, and/or other rating associated with the user account;
a type and/or an aspect of the procedure;
a type of the patient;
or any combination thereof.

23. The system of claim 21, wherein the authorization level associated with the user account is selected from one of the following authorization levels:
a first authorization level at which the user account is restricted from modifying at least one of an injection protocol and a scanning protocol;
a second authorization level at which the user account is restricted from modifying a first subset of parameters of the at least one of the injection protocol and the scanning protocol and enabled to modify a second subset of parameters of the at least one of the injection protocol and the scanning protocol;
a third authorization level at which the user account is enabled to modify each parameter of the at least one of the injection protocol and the scanning protocol.

24. A method comprising:
providing, with at least one processor, a user interface;
receiving, with the at least one processor, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system;
providing, with the at least one processor, via the user interface, a display, wherein the display is generated based on the procedure data;
receiving, with the at least one processor, via the user interface, user input; and
controlling, with the at least one processor, based on the user input, an operation of at least one of the injection system and the imaging system.

25. A computer program product comprising at least one non-transitory computer-readable medium including program instructions that, when executed by at least one processor, cause the at least one processor to:
provide a user interface;
receive, from at least one of an injection system including an injector configured to deliver fluid to a patient and an imaging system including a scanner configured to scan the patient, procedure data associated with a procedure for the patient, wherein at least one of the injection system and the imaging system includes at least one further user interface different than the user interface, and wherein the injection system is in communication with the imaging system;
provide, via the user interface, a display, wherein the display is generated based on the procedure data;
receive, via the user interface, user input; and
control, based on the user input, an operation of at least one of the injection system and the imaging system.
